(19) United States
(12) Patent Application Publication
(10) Pub. No.: US 2026/0207161 A1
(43) Pub. Date: Jul. 23, 2026

(54) AUTOMATIC OPTIMAL X-RAY EMITTER POSITION DETECTION FOR MOBILE X-RAY DEVICES

(71) Applicant: KONINKLIJKE PHILIPS N.V., EINDHOVEN (NL)

(72) Inventors: ILYAS SIRAZITDINOV, HAMBURG (DE); JOËL VALENTIN STADELMANN, MOSCOW (RU)

(21) Appl. No.: 19/139,577

(22) PCT Filed: Dec. 13, 2023
(86) PCT No.: PCT/EP2023/085463

§ 371 Date: Jun. 16, 2025

(30) Foreign Application Priority Data
Dec. 22, 2022 (RU) 2022133850

(51) Int. Cl. A61B 6/58; A61B 6/00

(52) U.S. Cl. CPC A61B 6/589; A61B 6/4405; A61B 6/545; A61B 6/547

(57) ABSTRACT

In order to acquire mobile X-ray images of better quality, a computer-implemented method is provided for determining a position of an X-ray emitter of a mobile X-ray device. The method comprises the following steps: receiving (210) (i) a camera-acquired image originating from a camera that monitors a patient in an X-ray imaging session, wherein the camera is registered to an origin of the X-ray emitter, (ii) a reference X-ray image of an internal body structure to be examined in the X-ray examination, and (iii) position information of an X-ray detector; detecting (220) and localizing a plurality of anatomical landmarks in the camera-acquired image; determining (230) a distance from the X-ray emitter to the patient based on the camera-acquired image; generating (240) a virtual X-ray image of the internal body structure based on the plurality of detected anatomical landmarks, the distance from the X-ray emitter to the patient, and the position information of the X-ray detector; registering (250) the virtual X-ray image of the internal body structure with the reference X-ray image of the internal body structure; and determining (260) at least one parameter to adjust the position of the X-ray emitter based on a result of registration.

### FIELD OF THE INVENTION

The present invention relates to a computer-implemented method for determining a position of an X-ray emitter of a mobile X-ray device, to an X-ray emitter position determination device, to an X-ray imaging system, to a computer program product, and to a computer-readable medium.

### BACKGROUND OF THE INVENTION

Mobile X-ray, also referred to as portable X-ray, is an important image acquisition modality. It allows to take X-ray in places where stationary X-ray imaging is not practical, e.g., emergency department (ED), intensive care unit (ICU), etc. Mobile X-ray targets mainly patients lying in bed. In contrast to stationary X-ray, acquiring an X-ray image on portable X-ray more challenging since there are more degrees of freedom, than for stationary X-ray image.

FIG. 1 shows the basic working principle of a mobile X-ray machine 10. First, the technician places an X-ray detector 12 behind a patient, shown as bed-patient in FIG. 1. Then, the technician manually sets the position of an X-ray emitter 14 so that the desired field of view can be captured by the X-ray detector 12. Finally, the technician acquires an image by activating the X-ray emitter 14. There are a plenty of parameters that can seriously affect the image quality, such as the position of X-ray detector 12 (e.g., coordinates and rotation angles) and the position of the X-ray emitter (e.g., distance to patient, rotation angles, and voltage).

### SUMMARY OF THE INVENTION

Thus, there may be a need to acquire mobile X-ray images of better quality.

The object of the present invention is solved by the subject-matter of the appended independent claims, wherein further embodiments are incorporated in the dependent claims.

According to a first aspect of the present invention, there is provided a computer-implemented method for determining a position of an X-ray emitter of a mobile X-ray device, comprising:
a) receiving (i) a camera-acquired image originating from a camera that monitors a patient in an X-ray imaging session, wherein the camera is registered to an origin of the X-ray emitter, (ii) a reference X-ray image of an internal body structure to be examined in the X-ray examination, and (iii) position information of an X-ray detector;
b) detecting and localizing a plurality of anatomical landmarks in the camera-acquired image;
c) determining a distance from the X-ray emitter to the patient based on the camera-acquired image;
d) generating a virtual X-ray image of the internal body structure based on the plurality of detected anatomical landmarks, the distance from the X-ray emitter to the patient, and the position information of the X-ray detector;
e) registering the virtual X-ray image of the internal body structure with the reference X-ray image of the internal body structure; and
f) determining at least one parameter to adjust the position of the X-ray emitter based on a result of registration.

In other words, the present disclosure proposes a method that can automatically locate the optimal X-ray emitter position for an image using either a reference X-ray image, such as a prior high-quality image from the same patient or a reference image from an atlas, deemed suitable for this imaging case. With the method disclosed herein, a reduction of the radiation dose received by the patients may be achieved by lowering the number of retakes. Additionally, reduced turnaround times may be achieved by avoiding low quality images being send to the PACS and rejected at the time of their review. Furthermore, because of the multiple job repetitions may be reduced, the costs may also be reduced.

This will be explained in detail hereinafter and in particular with respect to the examples shown in FIG. 3.

According to an embodiment of the present invention, the step of generating a virtual X-ray image of the internal body structure further comprises:
producing a pseudo density image of the patient based on the plurality of detected anatomical landmarks; and
projecting the pseudo density image of the patient to the X-ray detector based on a camera position of the camera, the distance from the X-ray emitter to the patient, and position information of the X-ray detector using a cone-beam projection to obtain the virtual X-ray image of the internal body structure.

This will be explained in detail hereinafter and in particular with respect to the example shown in FIG. 5.

According to an embodiment of the present invention, the camera comprises a three-dimensional (3D) camera and/or a two-dimensional (2D) camera. The distance from the X-ray emitter to the patient is determined based on depth information in the camera-acquired image or a neural network based depth estimation.

This will be explained in detail hereinafter and in particular with respect to step 230 shown in FIG. 3.

According to an embodiment of the present invention, the step of registering the virtual X-ray image of the internal body structure with the reference X-ray image of the internal body structure further comprises:
applying a pre-trained neural network to register the virtual X-ray image of the internal body structure with the reference X-ray image of the internal body structure, wherein the pre-trained neural network has been trained to generate a residual parameter of a camera position of the camera from the virtual X-ray image, the reference X-ray image, and the plurality of detected anatomical landmarks, wherein the residual parameter of the camera position is usable to transform the virtual X-ray image to the reference X-ray image.

This will be explained in detail hereinafter and in particular with respect to the example shown in FIG. 3.

According to an embodiment of the present invention, the at least one parameter for adjusting the position of the X-ray emitter includes one or more of:
a parameter for adjusting the distance from the X-ray emitter to the patient; and
a parameter for adjusting a rotation angle of the X-ray emitter.
   According to an embodiment of the present invention, the computer-implemented method further comprises:
providing, based on the at least one determined parameter, an instruction signal to guide a user to manually adjust the position of the X-ray emitter.

According to an embodiment of the present invention, the computer-implemented method further comprises:
providing, based on the at least one determined parameter, a control signal usable to control a mobile X-ray robotic arm of the mobile X-ray device to adjust the position of the X-ray emitter.

According to an embodiment of the present invention, the reference X-ray image comprises one or more of:
a patient's previously acquired X-ray image of the internal body structure; and
a reference X-ray image from an atlas.

According to a second aspect of the present invention, there is provided an X-ray emitter position determination device comprising a processing unit configured to perform the steps of the method according to the first aspect and any associated example.

This will be explained in detail hereinafter and in particular with respect to the example shown in FIG. 2.

According to a third aspect of the present invention, there is provided an X-ray imaging system, comprising:
a mobile X-ray device comprising an X-ray emitter;
a camera mountable to the mobile X-ray device and configured to capture a camera-acquired image from a patient in an X-ray imaging session, wherein the camera is registered to an origin of the X-ray emitter;
an X-ray detector;
a tracker device that is attachable to the X-ray detector or embedded in the X-ray detector, and configured to provide position information of the X-ray detector; and
the X-ray emitter position determination device according to the second aspect and any associated example configured to determine at least one parameter to adjust a position of the X-ray emitter.

This will be explained in detail hereinafter and in particular with respect to the example shown in FIG. 2.

According to an embodiment of the present invention, the tracker device comprises one or more of: a marker device, a gyroscope, and an antenna.

According to an embodiment of the present invention, the mobile X-ray device further comprises a mobile X-ray robotic arm to support the X-ray emitter. The X-ray emitter position determination device is configured to provide a control signal to control the mobile X-ray robotic arm of the mobile X-ray device to adjust the position of the X-ray emitter.

According to an embodiment of the present invention, the mobile X-ray device further comprises a display configured to display an instruction provided by the X-ray emitter position determination device to guide a user to manually adjust the position of the X-ray emitter.

The predicted directions are used on the X-ray machine: they either can be displayed for the manual correction by the technician, or an automated correction by a mobile X-ray robotic arm (if present) can be realized.

According to another aspect of the present invention, there is provided a computer program product comprising instructions which, when the program is executed by a processing unit, cause the processing unit to carry out the steps of the method disclosed herein.

According to a further aspect of the present invention, there is provided a computer-readable medium having stored thereon the computer program product.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 shows a basic working principle of a mobile X-ray machine.

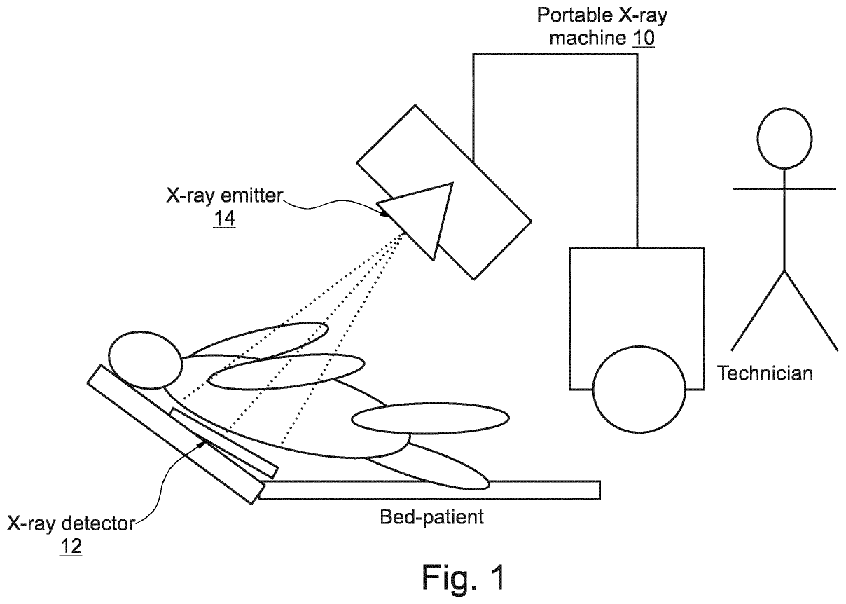

FIG. 2 illustrates an exemplary X-ray imaging system.

FIG. 3 illustrates a flowchart describing a computer-implemented method for determining a position of an X-ray emitter of a mobile X-ray device.

FIG. 4 illustrates an exemplary registration process.

FIG. 5 shows a flow diagram describing the generation of a virtual X-ray image of the internal body structure, according to an implementation.

FIG. 6 shows an exemplary working principle of the exemplary X-ray imaging system shown in FIG. 2

### DETAILED DESCRIPTION OF EMBODIMENTS

In clinical practice, mobile X-ray systems may be limited in terms of image quality compared to their stationary counterparts, i.e., bad field of view, missed or cut anatomy. Bad images may lead to image retakes, which increase the radiation dose for the patient.

Towards this end, a method, apparatus, and system are provided with the aim of improving the image quality and overcoming one or more of the above-mentioned problems of mobile X-ray during the image acquisition stage. In particular, a system is proposed that can automatically acquire mobile X-ray images of better quality. The system disclosed herein may automatically locate the optimal X-ray emitter position for an image using either a prior high-quality image from the same patient, or a reference image from an atlas, deemed suitable for this imaging case.

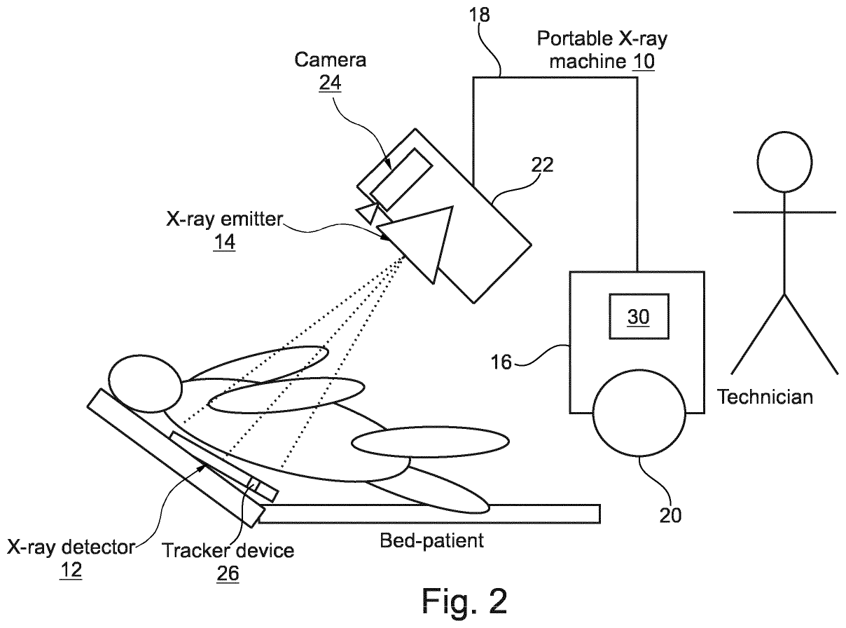

FIG. 2 illustrates an exemplary X-ray imaging system 100 according to an embodiment of the present invention. The X-ray imaging system 100 comprises a mobile X-ray device 10 with an X-ray emitter 14. As shown in FIG. 2, the mobile X-ray device 10 further comprises a chassis 16 that supports an arm, e.g., a robotic arm 18, and having a system with wheels 20 for manual or motorized movement which allow the equipment to be transported. The robotic arm 18 may move horizontally and/or vertically and support at its end a head assembly 22, where the X-ray emitter 14 is located.

The X-ray imaging system 100 further comprises a camera 24 mountable to the mobile X-ray device 10 and configured to capture a camera-acquired image from a patient in an X-ray imaging session. The camera 24 is registered to an origin of the X-ray emitter 14. For example, as shown in FIG. 2, the camera 24 may be located in the head assembly 22. In some examples, the camera 24 may be an embedded camera. In some other examples, the camera 24 may be removably mounted to the head assembly 22. In some examples, the camera 24 may be a two-dimensional (2D) camera configured for capturing one scene by using one photographing lens and one image sensor. The obtained image is called a 2D image. In some examples, the camera 24 may be a three-dimensional (3D) camera for capturing 3D images. The 3D camera may be a range camera, which produces a 2D image showing the distance to points in a scene from a specific point. The 3D camera may be e.g., a stereo camera, which is a type of camera with two or more lenses with separate image sensors or film frame for each lens.

The X-ray imaging system 100 shown in FIG. 2 further comprises a portable X-ray detector 12, which may be arranged behind a patient in order to measure the flux, spatial distribution, spectrum, and/or other properties of X-rays. The portable X-ray detector 12 may be fitted with a tracker device 26, such as a gyroscope, antennas, a marker or other devices, which help to localize the portable X-ray detector's position relative to the X-ray emitter 14.

The X-ray imaging system 100 shown in FIG. 2 further comprises an X-ray emitter position determination device 30 configured to determine at least one parameter to adjust a position of the X-ray emitter 14. In general, the X-ray emitter position determination device 30 may comprise various physical and/or logical components for communicating and manipulating information, which may be implemented as hardware components (e.g., computing devices, processors, logic devices), executable computer program instructions (e.g., firmware, software) to be executed by various hardware components, or any combination thereof, as desired for a given set of design parameters or performance constraints.

In some implementations, the X-ray emitter position determination device 30 may be embodied as, or in, a device, such as the mobile X-ray device 10 shown in FIG. 2 or mobile device. The X-ray emitter position determination device 30 may comprise one or more microprocessors or computer processors, which execute appropriate software. The processing unit of the apparatus 10 may be embodied by one or more of these processors. The software may have been downloaded and/or stored in a corresponding memory, e.g., a volatile memory such as RAM or a non-volatile memory such as flash. The software may comprise instructions configuring the one or more processors to perform the functions as described herein.

It is noted that the X-ray emitter position determination device 30 may be implemented with or without employing a processor, and also may be implemented as a combination of dedicated hardware to perform some functions and a processor (e.g., one or more programmed microprocessors and associated circuitry) to perform other functions. For example, the functional units of the X-ray emitter position determination device 30 may be implemented in the device or apparatus in the form of programmable logic, e.g., as a Field-Programmable Gate Array (FPGA). In general, each functional unit of the apparatus may be implemented in the form of a circuit.

Although FIG. 2 may show that the X-ray emitter position determination device 30 is embodied in the mobile X-ray device 10, it will be appreciated that in some implementations the X-ray emitter position determination device 30 may be embodied as, or in, a mobile device, e.g., tablet computer.

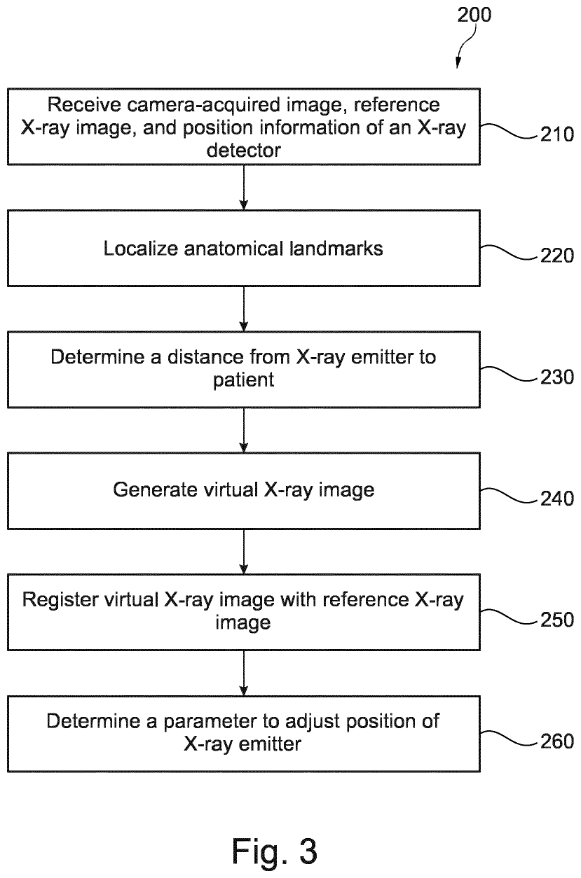

The X-ray emitter position determination device 30 is configured to perform the method disclosed herein. The method will be described in detail hereinafter and in conjunction with the flowchart shown in FIG. 3.

FIG. 3 illustrates a flowchart describing a computer-implemented method 200 for determining a position of an X-ray emitter of a mobile X-ray device. The method 200 may be implemented as a device, module or related component in a set of logic instructions stored in a non-transitory machine- or computer-readable storage medium such as random access memory (RAM), read only memory (ROM), programmable ROM (PROM), firmware, flash memory, etc., in configurable logic such as, for example, programmable logic arrays (PLAs), field programmable gate arrays (FPGAs), complex programmable logic devices (CPLDs), in fixed-functionality hardware logic using circuit technology such as, for example, application specific integrated circuit (ASIC), complementary metal oxide semiconductor (CMOS) or transistor-transistor logic (TTL) technology, or any combination thereof. For example, computer program code to carry out operations shown in the method 200 may be written in any combination of one or more programming languages, including an object oriented programming language such as JAVA, SMALLTALK, C++, Python, or the like and conventional procedural programming languages, such as the “C” programming language or similar programming languages. For example, the exemplary method may be implemented as the apparatus 30 shown in FIG. 2.

In step 210, the method 200 comprises a step of receiving (i) a camera-acquired image originating from a camera that monitors a patient in an X-ray imaging session, wherein the camera is registered to an origin of the X-ray emitter, (ii) a reference X-ray image of an internal body structure to be examined in the X-ray examination, and (iii) position information of an X-ray detector.

The camera-acquired image may originate from the camera 24 shown in FIG. 2 that monitors the patient in an X-ray imaging session. In some examples, the camera 24 may be a video camera configured to send the real-time video stream to the apparatus 30, which may have a video-processing unit to process the real-time video stream. In some examples, the camera 24 may be a camera configured to capture images and send the images to the apparatus 30, which may have an image-processing unit to process the images. In some examples, the camera-acquired image may comprise one or more 3D images. In some examples, the camera-acquired image may comprise one or more 2D images.

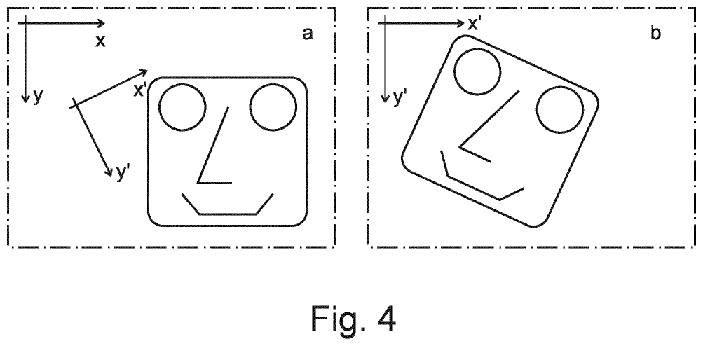

The camera 24 is registered to an origin of the X-ray emitter 14. FIG. 4 illustrates an exemplary registration process. When an object is imaged, its representation is stored in a matrix of pixels, which can be addressed by their coordinates x, y. Usually, the origin (i.e., the 0, 0 point) is located in the upper left corner of the matrix, with the x axis going from left to right, and the y axis from top to bottom. Unless the object and the camera are rigidly attached to each other, imaging the object twice will result in two different matrices, with two different coordinate systems. In the example of FIG. 4, the nose of the patient on image “a” is located further on the right and down, than on image “b”. It is possible to register the two images together to evaluate the coordinate transformation, which permits to transform one image into the other. Since the imaging planes is perpendicular to the axis of the camera, the transformation of the images directly translates into the camera movements required to reacquire one image if the second is available.

In the present disclosure it is proposed to use image registration in two separated instances:
The transformation from the video camera to the emitter coordinate systems is constant and may be expressed explicitly when designing the relative positions of the two objects; and
The transformation between the positions of the emitter during two different imaging session is likely to be more complicated, than the translation and rotation shown on FIG. 4. Therefore, it is proposed to learn this transformation using a neural network, e.g., a convolutional network. This will be explained in detail hereinafter.

The reference X-ray image of an internal body structure to be examined in the X-ray examination may be downloaded from a Picture Archiving and Communication System (PACS). The reference X-ray image may be a previous scan of the patient of a good quality or some reference image or atlas of a good quality. In some examples, the reference X-ray image may be acquired from a different patient.

The position information of an X-ray detector 12 may be acquired from the tracker device 26, e.g., a marker device, a gyroscope, an antenna, or any combination thereof, which is attached to the X-ray detector 12.

In step 220, the method 200 further comprises a step of detecting and localizing a plurality of anatomical landmarks in the camera-acquired image. In some examples, a model of the patient may be determined prior to imaging in order to conform the imaging parameters to the patient anatomy. The model may include locations of anatomical landmarks, such as shoulders, pelvis, torso, knees, etc. An acquired surface image of a patient may be compared against a library of pre-modeled surface images to determine a model corresponding to the patient. The determination may be performed by a neural network which is trained based on the library of pre-modeled surface images. In some examples, the location of a head, shoulder, torso, knee, and ankle may be determined based on a 2D or 3D image. Neural networks may be trained to detect the landmarks automatically. For example, supervised learning can be applied for anatomical landmark localization. See for example Gite, S., Mishra, A., & Kotecha, K. (2022). Enhanced lung image segmentation using deep learning. Neural Computing and Applications, 1-15.

In step 230, the method 200 further comprises a step of determining a distance from the X-ray emitter to the patient based on the camera-acquired image. If the camera is a 3D camera, the distance from the X-ray emitter to the patient may be determined based on depth information in the camera-acquired image. If the camera is a 2D camera, the distance from the X-ray emitter to the patient may be determined based on a neural network based depth estimation. For example, self-supervised learning may be applied for the distance or depth estimation. See for example Bian, J., Li, Z., Wang, N., Zhan, H., Shen, C., Cheng, M. M., & Reid, I. (2019). Unsupervised scale-consistent depth and ego-motion learning from monocular video. Advances in neural information processing systems, 32.

In step 240, the method 200 further comprises a step of generating a virtual X-ray image of the internal body structure based on the plurality of detected anatomical landmarks, the distance from the X-ray emitter to the patient, and the position information of the X-ray detector.

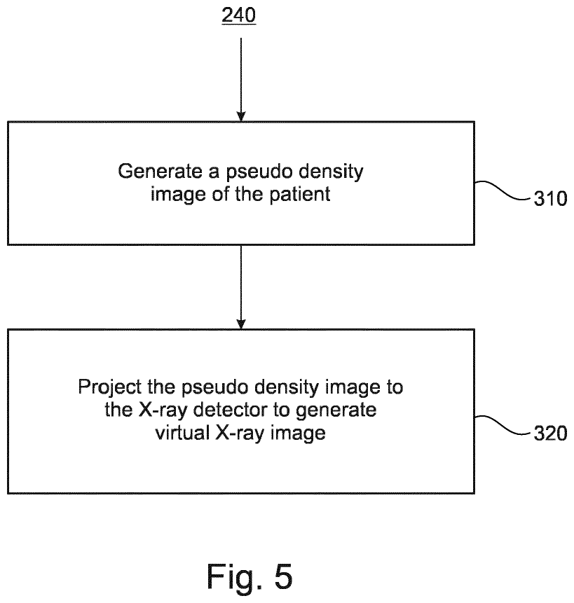

FIG. 5 shows a flow diagram describing one implementation of step 240.

In step 310 of step 240, a pseudo density image of the patient is generated based on the plurality of detected anatomical landmarks.

In step 320 of step 240, the pseudo density image of the patient is projected to the X-ray detector based on a camera position of the camera, the distance from the X-ray emitter to the patient, and position information of the X-ray detector using a cone-beam projection to obtain the virtual X-ray image of the internal body structure.

In other words, a virtual X-ray image may be generated in two steps. Frist, anatomical landmarks are used to produce a pseudo density image of the patient e.g., by a neural network. Such network can be trained using annotated pairs of images, e.g., CT images and the corresponding masks. The neural network may be a convolutional network. Adversarial training may be used to train this model. See for example Park, T., Liu, M. Y., Wang, T. C., & Zhu, J. Y. (2019). Semantic image synthesis with spatially-adaptive normalization. In Proceedings of the IEEE/CVF conference on computer vision and pattern recognition (pp. 2337-2346). The resulted pseudo density image is then projected to the detector given the camera position, distance to patient, and the relative detector position using cone-beam projection. The obtained image is a virtual X-ray image.

Turning back to FIG. 3, in step 250, the method 200 further comprises a step of registering the virtual X-ray image of the internal body structure with the reference X-ray image of the internal body structure

In some examples, a pre-trained neural network may be applied to register the virtual X-ray image of the internal body structure with the reference X-ray image of the internal body structure. The pre-trained neural network has been trained to generate a residual parameter of a camera position of the camera from the virtual X-ray image, the reference X-ray image, and the plurality of detected anatomical landmarks. The residual parameter of the camera position is usable to transform the virtual X-ray image to the reference X-ray image. For example, a neural network may have been trained in the following way. The neural network takes three inputs: a reference X-ray image, a virtual X-ray image, and an anatomical landmarks map. The output of the model is a residual parameter of the camera position. Such parameters can be applied to transform virtual image towards reference image. The required images for training are artificially sampled from pseudo CT projects using static detector parameters and flexible emitter. The registration results are used to predict the directions on how to adjust the emitter's position. The neural network may be a convolutional network. Self-supervised learning can be applied to train this model. See for example Dalca, A. V., Balakrishnan, G., Guttag, J., & Sabuncu, M. R. (2018, September). Unsupervised learning for fast probabilistic diffeomorphic registration. In International Conference on Medical Image Computing and Computer-Assisted Intervention (pp. 729-738). Springer, Cham.

In step 260, the method 200 further comprises a step of determining at least one parameter to adjust the position of the X-ray emitter based on a result of registration. In other words, the registration results are used to predict the directions on how to adjust the emitter's position. The at least one parameter may include one or more of a parameter for adjusting the distance from the X-ray emitter to the patient and a parameter for adjusting a rotation angle of the X-ray emitter.

In some examples, the apparatus 30 may provide, based on the at least one determined parameter, an instruction signal to guide a user to manually adjust the position of the X-ray emitter. In some examples, the instruction signal may be a voice signal for guiding a technician to manually correct the position of the X-ray emitter. In some examples, the instruction signals may be a displayed instruction used to guide a technician to manually correct the position of the X-ray emitter. In some other examples, the apparatus 30 may provide based on the at least one determined parameter, a control signal usable to control a mobile X-ray robotic arm 18 of the mobile X-ray device 10 to adjust the position of the X-ray emitter 14.

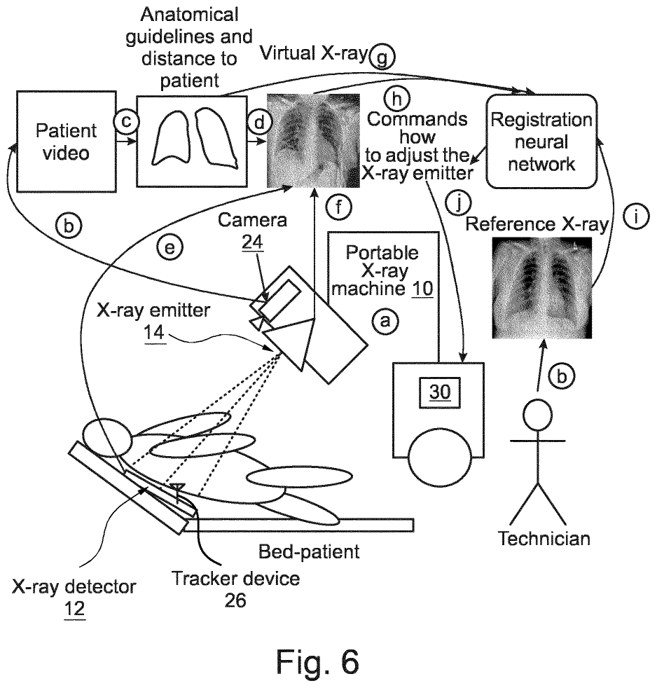

FIG. 6 shows an exemplary working principle of the exemplary X-ray imaging system 100 shown in FIG. 2.

The mobile X-ray machine 10 is placed in front of the patient (indicated with “a”). The X-ray detector 12 is placed behind the patient.

The technician may download from the PACS a reference X-ray image (indicated with “b”). It may be a previous scan of a good quality or some reference image or atlas of a good quality. The reference X-ray image is provided to the apparatus 30. The camera 24 sends a camera-acquired image, e.g., the real-time video stream or images, to the apparatus 30.

The apparatus 30 may comprise a first neural network, also referred to as neural network A, to predict anatomical landmarks and the distance to the patient (indicated with “c”) based on the camera-acquired image in the patient video.

The apparatus 30 may comprise a second neural network, also referred to as neural network B, to predict a virtual X-ray image using the anatomical landmarks (indicated with “d”) and the X-ray emitter (indicated with “f”) and detector position (indicated with “e”). A virtual X-ray image is generated in two steps. 3D anatomical landmarks from neural network A are being used to produce a pseudo density image of the patient by neural network B. Such network can be trained using annotated pairs of images, e.g., CT images and the corresponding masks. The resulted pseudo density image is being projected to the detector given the camera position, distance to patient, and the relative detector position using cone-beam projection. The obtained image is a virtual X-ray image.

The apparatus 30 may comprise a third neural network, also referred to as neural network C, to register the virtual X-ray (indicated with “h”) with the reference X-ray image (indicated with “i”) using the anatomical landmarks (indicated with “g”). The neural network C has been trained in the following way. It takes three inputs: a reference X-ray image, a virtual X-ray image and an anatomical landmarks map. The output of the model is a residual parameter of the camera position. Such parameters can be applied to transform virtual image towards reference image. The required images for training are artificially sampled from pseudo CT projects using static detector parameters and flexible emitter parameters. The registration results are used to predict the directions on how to adjust the emitter's position.

The predicted directions (indicated with “j”) are used on the X-ray machine: they either can be displayed for the manual correction by the technician, or an automated correction by a mobile X-ray robotic arm (if present) can be realized.

The method, apparatus, and system as disclosed herein can automatically locate an optimal X-ray emitter position for an image using either a prior high-quality image from the same patient, or a reference image from an atlas, deemed suitable for this imaging case, and can automatically acquire mobile X-ray images of better quality. The method, apparatus, and system as disclosed herein may lower the number of retakes and therefore achieve a reduction of the radiation dose received by the patients. The method, apparatus, and system as disclosed herein may avoid low quality images being sent to the PACs and rejected at the time of the review and therefore achieve reduced turnaround times. The method, apparatus, and system as disclosed herein may also reduce costs, because of the multiple job repetitions may be reduced.

In another exemplary embodiment of the present invention, a computer program or a computer program element is provided that is characterized by being adapted to execute the method steps of the method according to one of the preceding sfs45kk, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

1. A computer-implemented method for determining a position of an X-ray emitter of a mobile X-ray device, comprising:
receiving (i) a camera-acquired image originating from a camera that monitors a patient in an X-ray imaging session, wherein the camera is registered to an origin of the X-ray emitter, (ii) a reference X-ray image of an internal body structure to be examined in the X-ray examination, and (iii) position information of an X-ray detector;
detecting and localizing a plurality of anatomical landmarks in the camera-acquired image;
determining a distance from the X-ray emitter to the patient based on the camera-acquired image;
generating a virtual X-ray image of the internal body structure based on the plurality of detected anatomical landmarks, the distance from the X-ray emitter to the patient, and the position information of the X-ray detector;
registering the virtual X-ray image of the internal body structure with the reference X-ray image of the internal body structure; and
determining at least one parameter to adjust the position of the X-ray emitter based on a result of registration.

2. The computer-implemented method according to claim 1, wherein generating the virtual X-ray image of the internal body structure based on the plurality of detected anatomical landmarks further comprises:
producing a pseudo density image of the patient based on the plurality of detected anatomical landmarks; and
projecting the pseudo density image of the patient to the X-ray detector based on a camera position of the camera, the distance from the X-ray emitter to the patient, and position information of the X-ray detector using a cone-beam projection to obtain the virtual X-ray image of the internal body structure.

3. The computer-implemented method according to claim 1, wherein the camera comprises a three-dimensional camera and a two-dimensional camera; and wherein the distance from the X-ray emitter to the patient is determined based on depth information in the camera-acquired image or a neural network based depth estimation.

4. The computer-implemented method according to claim 1, wherein registering the virtual X-ray image of the internal body structure with the reference X-ray image of the internal body structure further comprises:
applying a pre-trained neural network to register the virtual X-ray image of the internal body structure with the reference X-ray image of the internal body structure, wherein the pre-trained neural network has been trained to generate a residual parameter of a camera position of the camera from the virtual X-ray image, the reference X-ray image, and the plurality of detected anatomical landmarks, wherein the residual parameter of the camera position is usable to transform the virtual X-ray image to the reference X-ray image.

5. The computer-implemented method according to claim 1, wherein the at least one parameter for adjusting the position of the X-ray emitter includes one or more of:
a parameter for adjusting the distance from the X-ray emitter to the patient; and
a parameter for adjusting a rotation angle of the X-ray emitter.

6. The computer-implemented method according to claim 1, further comprising:
providing, based on the at least one determined parameter, an instruction signal to guide a user to manually adjust the position of the X-ray emitter.

7. The computer-implemented method according to claim 1, further comprising:
providing, based on the at least one determined parameter, a control signal usable to control a mobile X-ray robotic arm of the mobile X-ray device to adjust the position of the X-ray emitter.

8. The computer-implemented method according to claim 1, wherein the reference X-ray image comprises one or more of:
a patient's previously acquired X-ray image of the internal body structure; and
a reference X-ray image from an atlas.

9. (canceled)

10. An X-ray imaging system, comprising:
a mobile X-ray device comprising an X-ray emitter;
a camera mountable to the mobile X-ray device and configured to capture a camera-acquired image from a patient in an X-ray imaging session, wherein the camera is registered to an origin of the X-ray emitter;
an X-ray detector;
a tracker device that is attachable to the X-ray detector or embedded in the X-ray detector, and configured to provide position information of the X-ray detector; and
a processor configured to:
receive (i) a camera-acquired image originating from a camera that monitors a patient in an X-ray imaging session, wherein the camera is registered to an origin of the X-ray emitter, (ii) a reference X-ray image of an internal body structure to be examined in the X-ray examination, and (iii) position information of an X-ray detector;
detect and localize a plurality of anatomical landmarks in the camera-acquired image;
determine a distance from the X-ray emitter to the patient based on the camera-acquired image;
generate a virtual X-ray image of the internal body structure based on the plurality of detected anatomical landmarks, the distance from the X-ray emitter to the patient, and the position information of the X-ray detector;
register the virtual X-ray image of the internal body structure with the reference X-ray image of the internal body structure; and
determine at least one parameter to adjust the position of the X-ray emitter based on a result of registration.

11. The X-ray imaging system according to claim 10, wherein the tracker device comprises one or more of:
a marker device;
a gyroscope; and
an antenna.

12. The X-ray imaging system according to claim 10, wherein the mobile X-ray device further comprises a mobile X-ray robotic arm to support the X-ray emitter; and wherein the processor is configured to provide a control signal to control the mobile X-ray robotic arm of the mobile X-ray device to adjust the position of the X-ray emitter.

13. The X-ray imaging system according to claim 10, further comprising:
a display configured to display an instruction to guide a user to manually adjust the position of the X-ray emitter.

14. (canceled)

15. (canceled)

16. A non-transitory computer readable medium comprising executable instructions which, when executed by at least one processor, cause the at least one processor to perform a method for determining a position of an X-ray emitter of a mobile X-ray device, the method comprising:
receiving (i) a camera-acquired image originating from a camera that monitors a patient in an X-ray imaging session, wherein the camera is registered to an origin of the X-ray emitter, (ii) a reference X-ray image of an internal body structure to be examined in the X-ray examination, and (iii) position information of an X-ray detector;
detecting and localizing a plurality of anatomical landmarks in the camera-acquired image;
determining a distance from the X-ray emitter to the patient based on the camera-acquired image;
generating a virtual X-ray image of the internal body structure based on the plurality of detected anatomical landmarks, the distance from the X-ray emitter to the patient, and the position information of the X-ray detector;
registering the virtual X-ray image of the internal body structure with the reference X-ray image of the internal body structure; and
determining at least one parameter to adjust the position of the X-ray emitter based on a result of registration.
